(19) United States
(12) Patent Application Publication
(10) Pub. No.: US 2006/0110411 A1
(43) Pub. Date: May 25, 2006

(54) Streptococcus equi vaccine compositions and method of use

(75) Inventor: Hsien-Jue Chu, Fort Dodge, IA (US)

Correspondence Address: DARBY & DARBY P.C., P.O. BOX 5257, NEW YORK, NY, 10150-5257, US

(73) Assignee: Wyeth - Fort Dodge Laboratories, Fort Dodge, IA (US)

(21) Appl. No.: 11/325,556

(22) Filed: Jan. 3, 2006

Related U.S. Application Data
(63) Continuation of application No. 09/007,385, filed on Jan. 15, 1998.

(51) Int. Cl. A61K 39/09

(52) U.S. Cl. 424/244.1

(57) ABSTRACT

This invention relates to compositions comprising live, attenuated Streptococcus equi (S. equi), or a fractional extract of S. equi, in combination with at least one immunostimulant for stimulating mucosal immunity, such as saponin. The invention also relates to methods of preparation and dosage forms containing the composition of the invention as well as methods of use for stimulating the immune system of an equine and inducing an immune response to S. equi by contacting the cells of nasopharyngeal mucosa with the composition of the invention. Furthermore, the invention relates to a method of immunizing an equine to induce protective immunity against S. equi.

This application is a continuation of U.S. application Ser. No. 09/007,385, filed on Jan. 15, 1998, the disclosure of which is herein incorporated by reference in its entirety.

### FIELD OF THE INVENTION

This invention relates to compositions comprising live, attenuated Streptococcus equi (S. equi), or a fractional extract of S. equi, in combination with at least one immunostimulant for stimulating mucosal immunity, such as saponin. The invention also relates to methods of preparation of such a composition and methods of use for stimulating the immune system of an equine and inducing a protective immune response to S. equi by contacting the cells of nasopharyngeal mucosa with the composition of the invention. Furthermore, the invention relates to a method of immunizing an equine to induce protective immunity against S. equi.

### BACKGROUND OF THE INVENTION

S. equi causes strangles, an acute upper respiratory tract disease of horses. This highly contagious disease is characterized by fever, nasal discharge and abscess formation in the retropharyngeal and mandibular lymph nodes. The swelling of the lymph nodes is frequently so severe that the animal airways become obstructed. Morbidity is generally high and can be as high as 100% in susceptible populations.

Horses infected with strangles (in the field or experimentally), which recover from the disease become highly resistant to reinfection. In view of this fact, attempts have been made to develop an effective and safe vaccine against strangles. For example, vaccines prepared from bacterins of S. equi, or fractional extracts thereof, such as M protein-rich extracts, were developed. However, the existing vaccine compositions are not completely satisfactory. Some are relatively ineffective at providing protection against S. equi in the field and others have side effects. One of the problems with this line of research was that scientists tried to induce protection against S. equi by stimulating bactericidal antibodies in the blood serum of the horse.

Two groups of researches have reported that vaccination may require stimulation of the nasopharyngeal immune response using a live S. equi. Timoney et al. (U.S. Pat. No. 5,183,659) have prepared a composition adapted for nasal and oral administration which contained a non-encapsulated avirulent strain of S. equi suspended in Todd Hewit broth. However, this composition, although known for about ten years (according to the PCT International publication date of Jan. 29, 1987), has not resulted in a commercially useful vaccine composition. This is likely because the vaccine described in the '659 patent is a high-dose vaccine which is not cost effective and may be unsafe given the high dose (i.e., number of S. equi organisms) used. In addition, in order to ensure an appropriate dose level at the expiration date of the vaccine, an extra amount of the organism (usually at least one full log above the minimum dose) must be added to the vaccine. A high dose vaccine having this additional amount of S. equi creates even greater concern for safety.

Another group of researchers (EP 786,518) prepared a composition for nasal administration containing an encapsulated S. equi strain TW928 having an unidentified 1 kb deletion in its genome. This composition, however, was not tested for its effectiveness in horses. Therefore, there is still a need in the art for effective and safe vaccines against S. equi, particularly those that can be safely administered to young horses.

The present inventors have surprisingly discovered that a composition containing a combination of a live, attenuated S. equi strain (or a fractional extract of S. equi) and at least one immunomodulator has the property of being safe and stimulating an immune response in the nasopharyngeal mucosa of an equine. The composition can be used to provide protective immunity against infection by S. equi at relatively low doses.

### SUMMARY OF THE INVENTION

The present invention teaches a composition having a live, attenuated S. equi, or a fractional extract of S. equi, in combination with at least one immunomodulator for stimulating mucosal immunity, and methods for its preparation and use.

Accordingly, in one aspect, the invention provides a composition containing an immunomodulator for stimulating mucosal immunity, such as saponin, in combination with a live, attenuated, S. equi strain.

In another aspect, the invention provides a composition containing a combination of an immunomodulator, such as saponin, and a fractional extract of S. equi, which extract has the property of stimulating an immune response upon contacting the cells in the nasopharyngeal mucosa of an equine.

In yet another aspect, the invention provides for a composition containing a live, attenuated S. equi (or a fractional extract of S. equi), an immunomodulator and at least one other equine pathogen (or an antigenic material from such pathogen).

In a further aspect of the invention, dosage forms containing the composition of the invention suitable for administration to nasopharyngeal mucosa of an equine are provided.

In yet further aspect of the invention, a method is provided for eliciting an immune response in the nasopharyngeal mucosa of an equine by contacting the mucosa with the composition of the invention.

In yet other aspect of the invention, a method for protecting an equine against an infection by S. equi is provided.

### DETAILED DESCRIPTION OF THE INVENTION

All patents, patent applications, and other literature cited herein are hereby incorporated by reference in their entirety. In the case of inconsistencies, the present disclosure will prevail.

The invention relates to compositions comprising a live attenuated S. equi, or a fractional extract of S. equi, in combination with at least one immunostimulant for stimulating mucosal immunity. The composition may also contain a mixture of two or more attenuated S. equi strains.

An S. equi strain suitable for use in the present invention may be encapsulated or non-encapsulated, is avirulent, and has the ability to induce an immune response in an equine after administration via a mucosal membrane (i.e., it is antigenic). “Avirulent stain” is understood not to be able to cause strangles in horses and includes any strain that a person of skill in the art would consider safe for administering to a horse as a vaccine. For example, a strain causing minor clinical signs, including fever, serous or mucopurulent nasal discharge or ocular discharge, is within the scope of the present invention since such clinical signs are considered acceptable vaccine side effects.

Generally, the strain to be used in the present invention has gene mutations such as nucleotide substitutions, insertions and/or deletions in its genome which abrogate its ability to cause strangles. Antigenic determinants of such S. equi strain capable of eliciting an immune response against S. equi in the nasopharyngeal mucosa of an equine are not affected by these substitutions, deletions or insertions. However, a strain containing conservative nucleotide substitutions in the nucleotide region encoding such an antigenic determinant is within the scope of the invention, since “conservative” nucleotide substitutions do not change the amino acid sequence of an antigenic determinant. A strain containing amino acid substitutions in the antigenic determinants of attenuated S. equi is also within the scope of the invention, provided that such substitutions do not abrogate antigenicity. In one preferred embodiment, an attenuated S. equi strain contains substitutions, deletions and/or insertions outside the nucleotide sequence encoding the 41,000 mw fragment of M protein. In another preferred embodiment, an attenuated S. equi strain contains substitutions, deletions and/or insertions outside the nucleotide sequence encoding the antigenic determinant(s) of the 41,000 mw fragment of M protein.

Live, attenuated (encapsulated and non-encapsulated) S. equi can be obtained from any virulent form of S. equi by using methods known in the art. For example, U.S. Pat. No. 5,183,659 to Timoney describes a method for producing non-encapsulated attenuated strains of S. equi. Briefly, a virulent strain of S. equi (for example, CF32, which is publicly available from American Type Culture Collection ATCC No. 53185) is subjected to nitrosoguanine mutagenesis, for example, as described in Gene Mutation, Chapter 13, Manual of Methods of General Bacteriology, American Society for Microbiology, Washington, DC 1981. Non-encapsulated S. equi colonies are screened by testing for loss of virulence by intraperitoneal inoculation of mice. The following papers describe the amount of S. equi used for intraperitoneal inoculation of mice: Timoney, J. F., Characteristics of an R Antigen Common to Streptococcus equi and zooepidemicus, Cornell Vet. 76:49-60 (1986) (in which strain e23 had an LD50 of 5×10⁶ CFU (4 LD50=2×10⁷)); and Timoney, et al., Cloning and Sequence Analysis of a Protective M-like Protein Gene from Streptococcus equi subsp. zooepidemicus, Infection and Immunity, April 1995, p. 1440-1445 (in which strain CF32 had an LD50 of 3.5×10⁵ CFU (2 LD50=7×10⁵)).

An example of an attenuated non-encapsulated strain of S. equi that can be used in the invention is S. equi strain 709-27 (ATCC No. 53186). This avirulent strain originated from Cornell Research Foundation, Ithaca, N.Y.

Methods of recombinant DNA technology can also be used to engineer deletions, insertions and substitutions in the S. equi genome and produce attenuated strains. These methods are well known in the art and are described, for example, in Sambrook et al. (Molecular Cloning, A Laboratory Manual, 2nd ed., Cold Spring Harbor Laboratory Press, 1989). Obtained mutant strains can be screened for loss of virulence by intraperitoneal inoculation of mice as described above.

Furthermore, M protein gene or a fragment thereof may be introduced into a live vector (e.g. Salmonella, raccoon pox virus) or into a vector for a killed product (e.g. baculovirus) and used for intranasal vaccination of horses. Genes of other antigens having a property of stimulating mucosal immunity may be used in a similar way.

Fractional extracts of S. equi may also be used in the composition of the present invention. Fractional extracts are defined herein as extracts of S. equi or extracts of S. equi antigens carried or expressed by vectors commonly used for insertion of foreign genes that have the property of eliciting an immune response after contacting the mucosa of an equine. Such fractional extracts can be from attenuated or wild type S. equi and are, for example, those extracts that contain M protein fragments or at least the M protein fragment having a molecular weight of about 41,000 mw. S. equi culture supernatants are also within the meaning of the term “fractional extract.”

Fractional extracts can be prepared using methods well known in the art. For example, the acid extract of S. equi is isolated as described in the U.S. Pat. No. 5,183,659 and according to techniques described in a publication by R. C. Lancefield entitled “The Antigenic Complex of Streptococcus Hemolyticus I Demonstration of a Type Specific Substance in Extracts of Streptococcus Hemolyticus,” J. Exp. Med. 47:91.

In one embodiment of the invention, a fractional extract of S. equi contains at least one antigenic determinant of a 41,000 mw fragment of M protein. Such antigenic determinant can be obtained by any known means in the art, such as for example using protein purification techniques or chemical synthesis.

The composition of the present invention also contains at least one immunostimulant to stimulate mucosal immunity. In one preferred embodiment of the invention, saponin is the immunostimulant. Any saponin or saponin derivative can be used. Preferably, such saponin has lipophilic and hydrophilic regions and therefore can function as a surfactant and emulsifier. In one preferred embodiment, Quil A is used. Quil A is available from commercial sources such as Superfos (Copenhagen, Denmark). In the present composition, saponin is used in the amount of from about 1 to about 10 mg/ml, preferably from about 3 to about 7 mg/ml, and most preferably from about 4 to about 6 mg/ml. The preferred saponin concentrations are based on a 2 ml dosage suitable for administration to equine through mucosal routes but can be adjusted by a person of skill in the art to achieve a comparable level of saponin in any dosage volume suitable for administration.

Other immunomodulators, particularly those suitable for nasal administration, can be used in the composition of the invention. For example, metabolizable oils, interleukins, interferons, bacterial toxoids and adjuvants, carbopol, dextran derivatives (e.g. dextran sulfate and DEAE-Dextran), and dimethyldiocta-declammonium bromide (DDA) can be used. Metabolizable oil (e.g. squalane, squalene, peanut oil) are generally used in the amount of from about 5 to about 60% (v/v), preferably from about 5 to about 40% (v/v) and most preferably from about 5 to about 20% (v/v). Interleukins (e.g. interleukin 1, 2 and 12) or interferons (alpha, beta or gamma) are generally used in the amount of from about 1 to about 50 ,ug/ml, preferably from about 3 to about 20 μg/ml and most preferably from about 3 to about 10 μg/ml. Bacterial adjuvants (e.g. Corynebacterium-derived adjuvants such as Corynebacterium parvum; Propionibacterium-derived adjuvants such as Propionibacterium acne; Mycobacterium bovis such as Bacillus Calmette Guerin, or BCG) are generally used in the amount of from about 50 μg/dose to 50 mg/dose, preferably from about 100 μg/dose to 25 mg/dose and most preferably from about 250 μg/dose to 15 mg/dose. Bacterial toxins (eg. Choleria toxin subunit, E. coli heat labile toxin) are generally used in the amount of from about 10 to about 500 μg/ml, preferably from about 10 to about 250 μg/ml and most preferably from about 10 to about 100 μg/ml. Carbopol is generally used in the amount from about 0.01 to 10% (w/v), preferably from about 0.1 to 5% (w/v), and most preferably from about 0.5 to 2% (w/v). Dextran derivatives and DDA are generally used in the amount of from about 0.01 to 10% (w/v), preferably from about 0.1 to about 5% (w/v) and most preferably from about 0.5 to 2% (w/v). Combinations of more than one immunomodulator (e.g. Quil®-A in combination with DEAE-Dextran or interleukin) are also within the scope of the present invention.

The composition of the present invention may be an oil or water emulsion and may also contain one or more pharmaceutically acceptable stabilizers and carriers. Carriers suitable for use include saline, phosphate-buffered saline, Minimal essential media (MEM), or MEM with HEPES buffer. Stabilizers include but are not limited to sucrose, gelatin, peptone, and digested protein extracts, such as NZ-Amine or NZ-Amine AS.

The composition of the present invention may optionally contain at least one other equine pathogen or an antigenic material from such pathogen. Such pathogens include, for example, Equine Influenza Virus A1, Equine Influenza Virus A2, Equine Herpes Virus 1b, Equine Herpes Virus 1p, Equine Herpes Virus 4, Rabies, Equine Viral Arteritis, Encephalomyelitis EEE, Encephalomyelitis WEE, Encephalomyelitis VEE and Clostridia tetani (toxoid).

The compositions of the present invention are prepared using methods known in the art, such as for example by admixing S. equi bacteria or extracts with an immunomodulator and any other additional components. The composition may be freeze dried for prolonged storage and reconstituted in a diluent prior to use. Alternatively, live, avirulent S. equi may be freeze dried and the composition of the invention may be reconstituted by resuspending the freeze-dried S. equi in a diluent containing an immunostimulant.

Dosage forms suitable for administration of the present composition to nasopharyngeal mucosa of an equine are also within the scope of the invention. Examples of such dosage forms are inhalers, nebulizers and nasal atomizers. In one preferred embodiment, the dosage form contains a syringe with the composition of the invention and a cannula for administration into the horse nostrils.

A dosage form may contain the composition of the invention in a lyophilized form to be reconstituted prior to use with a separately provided diluent. A dosage form may also contain a lyophilized attenuated S. equi strain (or a fractional extract of S. equi) to be reconstituted prior to use with a separately provided diluent containing an immunostimulant of the invention. In one preferred embodiment, the dosage form contains, at the time of manufacture, a maximum dose of about 5×10⁸ or about 1×10⁹ CFU of an attenuated S. equi, such as for example strain 709-27. In another preferred embodiment, the dosage form contains, at the expiration date, a minimum viable S. equi count of about 3.4×10⁷ CFU/dose or about 1.7×10⁷ CFU/dose. In the most preferred embodiment, the dosage form contains at the time of release 1×10⁸ CFU of S. equi in a lyophilized cake and a diluent (water) containing an immunostimulant (e.g. saponin at 5 mg/dose).

The invention further provides a method for eliciting an immune response in the nasopharyngeal mucosa of an equine by contacting the mucosa with the composition of the invention.

It is believed that the composition stimulates a local immune response similar to that in the nasopharyngeal mucus of an equine recovered from strangles. Such immune response can be stimulated in vivo (by administering the composition to an equine).

In vivo stimulation of the nasopharyngeal immune response in a susceptible equine is done by intranasal or by mouth administration of the composition of the invention. In one embodiment of the invention, about 2 ml of the composition is administered per dose. However, it is within the skill of a person of skill in the art to adjust the amount of the composition to be administered per dose. Each dose contains attenuated S. equi in the amount effective at stimulating an immune response in the nasopharyngeal mucosa of an equine. Generally, the amount is from about 10⁵ to about 10¹¹ CFU, preferably from about 10⁶ to about 10¹⁰ CFU, and most preferably from about 10⁷ to about 10⁹ CFU. In another preferred embodiment, the effective amount is from about 10⁵ to about 10⁸ CFU per dose. The effective amount will generally depend on the age, health and immune status (eg. previous exposure, maternal antibody) of the equine. A suitable effective amount, including the minimum antigen level and appropriate quantity of immunostimulant(s) required for protection, can be routinely determined by those skilled in the art using, for example, a dose titration procedure described in Example 2.

The composition of the invention may be administered as described above to healthy horses of four month of age and older to induce protective immunity against virulent strains of S. equi. A second, booster administration may be given from about ten days to about six weeks after the first administration, preferably about two to about five weeks after, and most preferably about two to about four weeks after. The composition may be re-administered annually to ensure prolonged protection. Animals vaccinated with the composition of the invention demonstrate significant differences (at least p<0.05) in mortality, total clinical score, disease incidence and leukocytosis following S. equi challenge in comparison to non-vaccinated animals.

The following non-limiting examples further describe the present invention.

### EXAMPLE 1

The objective of this study was to demonstrate the efficacy of the composition of the present invention against a virulent S. equi challenge.

Test Animals

Fifty-nine S. equi negative, clinically weanling Quarter horses were utilized in this study. The horses were screened by nasal isolation and ELISA against S. equi M-protein. Forty-nine horses were from Myrtle, Minn. and 10 horses were from Carpenter, Iowa. All horses were nine months old or younger at the time of the first vaccination. The horses were housed in an isolation facility for the duration of the study under veterinary care and were fed a standard commercial diet with water and food available ad libidum. The vaccinates and controls were housed separately during the vaccination period. All horses were housed together two days before challenge until the end of the study. The housing complied with applicable animal welfare regulations. No animals were treated with antibiotics or anti-inflammatory drugs during the duration of the study.

Vaccine Composition and Vaccination Schedule

The vaccine composition utilized in this study was prepared as follows. The vaccine strain was grown under controlled conditions in a fermenter to mid to late log phase. A pure culture of S. equi (master seed) was established and fully tested to meet government regulatory standards (9 C.F.R.). The culture was cooled and concentrated approximately 10× by hollow fiber filtration. The concentrate was mixed with SGGK stabilizer and lyophilized. The S. equi strain used in this study was produced at the highest passage level allowed from the master seed (i.e. MS+5). The normal production could be as low as MS +2 but by regulation can not exceed MS +5. Scientifically, higher passages than MS+5 could be used without adverse effect on the efficacy and safety of the vaccine. However, such use should always be evaluated by additional efficacy and safety studies.

The lyophilized S. equi preparation was reconstituted with deionized water containing 2.5 mg/mil of saponin (Berghausen Saponin). To obtain the target viable count adjustment was made by diluting the reconstituted vaccine with an adjustment diluent (75 percent Todd Hewitt broth, 25 percent SGGK stabilizer, and 2.5 mg/ml saponin). The vaccine was stored at 2-7° C. prior to use.

The horses were assigned at random to two test groups, Group A and Group B (each having 22 horses) and a Control Group of 15 horses. The randomization process was completed by random number assignment to each animal using Microsoft Excel. The numbers were sorted in ascending order.

Group A horses were administered two vaccine doses, 2 ml each. The first vaccine dose contained 3×10⁸ colony forming units (hereinafter “CFU”) of S. equi strain 709-27, and the second dose (administered three weeks later) contained 2×10⁸ CFU. Group B horses were also administered two vaccine doses, 2 ml each. The first vaccine dose contained 1.4×10⁷ CFU of S. equi strain 709-27, and the second dose (administered three weeks later) contained 1.7×10⁷ CFU. Control horses were not treated. The vaccines were titrated at the time of each vaccination.

Each vaccine dose was administered into a horse nostril using a five inch cannula. The first dose was administered into the left nostril and the second dose was administered into the right nostril 21 days after the first dose.

Challenge and Observation Procedure

Twenty-three days after the second vaccination, each of the 40 vaccinated and 15 control horses were challenged intranasally with a virulent S. equi organism (isolate CF32). Two horses from each Group A and Group B were removed from the study prior to challenge due to peritonitis resulting from multiple rectal perforations (caused by a temperature probe).

The challenge CF32 strain was used to inoculate modified Todd Hewitt Broth and the culture was grown at 37° C. on a rotary shaker at 150 rpm. The culture was harvested when an optical density of the culture reached 0.2 at a 1:10 dilution. The viable counts of the challenge culture were determined to be 7.4×10⁷ CFU/ml and 6.8×10⁷ CFU/ml prior to and post challenge, respectively. The challenge material was stored on ice before use. The challenge dose was administered at 1 ml per nostril inoculum.

The animals were observed daily from −2 days to 0 days post challenge (hereinafter “DPC”) to establish a baseline and 1 to 21 DPC for various clinical signs. Animals without ruptured lymph nodes were also observed on 25, 27, 32 and 35 DPC.

Nasal Swab Collection, Transport and Processing

Daily nasal swabs collected from −2 to 21 DPC were placed in 2 ml 0.01M PBS. The tubes were vortexed for 30 seconds and the swabs were removed taking care to express the liquid back into the tube. Serial ten fold dilutions were prepared in PBS and plated onto selective CNA agar plates (containing commercial CNA agar, Amphotericin B and Polymyxin B). The remaining fluid was stored at −70° C.

The plates were incubated for 36-48 hours at 37° C. ±2° C. and colonies with typical virulent S. equi morphology were counted. Representative suspect colonies were screened for ability to ferment lactose as a confirmatory measure. Typical colony morphology of challenge strain (CF-32) appeared to be translucent and mucoid with a large and clear B-hemolytic zone.

Whole Blood Samples and Hematology Evaluation

Five ml samples of whole blood were collected in EDTA-containing tubes daily, on −2 to 21 DPC, and analyzed by the Abbot Cell-dyne® blood counter for white blood cell count. Samples collected on 13, 15 and 16 DPC were not analyzed due to instrument failure. Baseline measurements for each horse were established as the average of the counts on −2 to 0 DPC.

Serum Collection and Antibody Evaluation Assays

Ten ml of whole blood was collected from each horse for serum preparation on 0, 7, 14 and 21 days after the first vaccination (DPV1), 7 and 14 days after the second vaccination (DPV2) and 0, 7, 14 and 21 days post challenge (DPC).

The sera were tested for antibodies against S. equi heat-extracted antigens by ELISA. A heat-extracted fraction containing M-protein was isolated from S. equi strain 709-27, according to the method described by Timoney et al., Infection & Immunity, 63(4): 1440-1445 (1995). The extract was used to coat the plates (0.02 μg per well) for measuring specific antibodies against S. equi. The sera were mixed with inactivated S. zooepidemicus to absorb cross reacting antibodies. The dilution of the serum following absorption was 1:160. Serial two fold dilutions of the sera were prepared in 0.01 M PBS. The dilution serum samples were added to duplicate wells of a coated plate and the plate was incubated for one hour at 37° C. Following washing, commercial anti-horse IgG conjugate was added to each well and the plate was incubated for one hour at 37° C. Substrate was added following washing and allowed to develop for 30 minutes at 37° C. Positive and negative control serum samples were run on each plate. The reaction was stopped by adding 1% SDS to each well and the absorbance was read at 490 nm in an automated microplate reader. A positive result was determined as an OD greater than or equal to 0.1 after calculating the sample to background ratio.

Clinical Scoring System

The following system was used to score clinical signs in challenged animals:

| (a) | Coughing | | (1 Point/Day) |
|---|---|---|---|
| (b) | Nasal discharge | | |
| | (1) | Serous | (1 Point/Day) |
| | (2) | Mucopurulent | (2 Points/Day) |
| (c) | Ocular discharge | | (1 Point/Day) |
| (d) | Depression | | (1 Point/Day) |
| (e) | Pyrexia | | (1 Point/degree above Baseline/Day*) |
| (f) | Labored breathing | | (2 Points/Day) |
| (g) | Enlargement of lymph nodes | | |
| | (1) | Head and neck areas | (2 Points/Day) |
| | (2) | Disseminated** | (3 Points/Day) |
| (h) | Abscesses of lymph nodes | | |
| | (1) | Head and neck areas | (25 Points/One Time Score) |
| | (2) | Disseminated | (40 Points/One Time Score) |
| | (i) | Death | (150 Points/One Time Score) |

*Must be greater than 103.0° F. to be considered as pyrexia
**Other than submandibular and pharyngeal lymph nodes.

Statistical Analysis

The level of significance for each statistical analysis was set at p<0.05. All analysis was completed on an IBM computer using SAS software. Mortality was compared using the Fiber Exact test. Total clinical score was compared using the Mann-Whitney U test. Swollen lymph node incidence and incidence of lymph node rupture were compared using Fishers Exact test. Daily hematology values (white blood cells) were compared by Analysis of Variance (GLM). Daily shedding incidence was compared by Fishers Exact Test. Antibody titers were compared using Analysis of Variance (GLM). Test Groups A and B were compared to the Control Group for each of the above tests.

### RESULTS

Clinical Observations

The daily clinical signs (from −2 DPC to 21 DPC) and daily rectal temperatures (from −2 DPC to 21 DPC) were observed. After S. equi challenge, all fifteen control horses showed severe clinical signs, including fever, depression, mucopurulent nasal discharge, coughing, labored breathing, enlarged lymph nodes and all the horses subsequently developed abscessed and ruptured lymph nodes. Two (2) control horses (#66 and #109) died on 15 and 21 DPC, respectively.

In contrast, eight of the vaccinates in group B were free of gross swelling and lymph nodes and two developed some swelling which did not progress towards rupture. Three of the vaccinates in group A were free of gross swelling lymph nodes and three developed some swelling which did not progress towards rupture.

Extended observations were made on 25, 27, 32, and 35 DPC to check additional lymph node swelling or ruptures and death of animals resulting from challenge. The swollen lymph nodes from the three horses (#3, #50 and #140) in group A regressed to normal at 35 DPC. One additional group B horse (#131) developed a swollen lymph node and one horse (#127) had a lymph node rupture.

The total number of horses that died to challenge or were euthanized for humane reasons (due to severe labored breathing or moribund state resulting from challenge) through the 35 days observation period were as follows: three of 20 horses (15%) from group A, two of 20 horses (10%) from group B, and nine of 15 horses (60%) from the controls. Staff veterinarians with no knowledge of treatment groups made the decision on animals requiring euthanasia. A significant difference in animals loss due to death or euthanization was demonstrated between both vaccinate groups and the controls group (p<0.05).

In the control group, 15 horses developed swollen lymph nodes and all 15 developed ruptured lymph nodes. In vaccinate group A, 17 horses developed swollen lymph nodes of which 14 ruptured. A significant difference was demonstrated between group A and the control group in the number of animals with ruptured lymph nodes throughout the 35 day observation period (p<0.05). In group B, 13 horses developed swollen lymph nodes of which 11 ruptured. A significant difference was demonstrated between group B and the control group in the number of animals with swollen lymph nodes as well as the number of animals with ruptured lymph nodes (p<0.05).

The total clinical scores of each group were obtained with an average score of 101 points (21 days post challenge) or 181 points (35 days post challenge) for the control group, 74.8 points (21 days post challenge) or 102.35 points (35 days post challenge) for the vaccinate group A and 59.3 points (21 days post challenge) or 76.35 points (35 days post challenge) for the vaccinate group B. A statistically significant difference was seen when comparing respective 21 or 35 days post challenge results in the total clinical scores of either vaccinate group to the control group (P<0.05).

The significant reduction in clinical score and disease incidence demonstrated that the vaccinated horses were significantly protected against clinical disease as compared to the controls following a severe S. equi challenge.

Total Peripheral White Blood Cell (WBC) Counts

The results of daily white cell counts and the daily average of each group were obtained. Three days following challenge, the mean group WBC began to increase (above baseline values). The average WBC count peaked at 8 DPC for both vaccinate groups with 23.0 (k/μl) for group A, 21.3(k/μl) for group B. The average WBC count peaked at 19 DPC for the control group with 30.7 (k/μl).

The average daily WBC count in the control group was consistently higher than that of both vaccinated groups throughout the observation period. Statistically, a significant difference (P<0.05) was seen when comparing daily WBC counts of the vaccinated group A on 5-8 DPC and the vaccinated group B on 4-8 DPC to the control group. A significant difference (P<0.05) was also shown when comparing daily WBC count of both vaccinated groups on 12-21 DPC to the control group.

Serological Responses

Serum IgG titer of the vaccinated and control horses were determined by the ELISA test. At 0 DPV1, all vaccinated horses has ELISA titers>1:160, except #47 (group A) with 1:320 and #39 (group B) with 1:640 (both were screened 19 days before first vaccination with ELISA titers >1:160). Horse #47 developed enlarged lymph nodes during the observation period, confirming susceptibility of these horses to challenge. Horses with ELISA titers as high as 1:640 were found to be susceptible to a S. equi challenge in previous preliminary studies. Twelve of the 15 control horses remained seronegative (ELISA titers>1:160) until 14 DPC when an elevated ELISA titer was detected. A minor increase in titer (most were less than 4 fold) was seen in the vaccinate groups from 0 DPV1 to 0 DPC. Statistically, neither of the vaccinated groups showed a significant difference in ELISA titer (serum IgG to S. equi) throughout the study when compared to the control (p>0.05). Serum ELISA titers have little value in predicting protection of susceptibility to challenge.

S. equi Shedding After Challenge

Following challenge, virulent S. equi was identified from most vaccinated and control horses from 1-9 DPC. After 10 DPC, as more horses developed abscesses, the incidence of shedding in both vaccinated groups and control group started to increase. A statistically significant difference was seen when comparing daily shedding incidence from 6 DPC to 7 DPC of vaccinate group B and 12 DPC of vaccinate group A to the control group (P<0.05).

Conclusion

The composition of the invention satisfactorily protects vaccinated horses against a severe virulent S. equi challenge. Statistically significant differences (at least P<0.05) between vaccinated groups and the control group are demonstrated in mortality, total clinical score, disease incidence and leukocytosis following S. equi challenge. The data demonstrate that the composition is immunogenic and efficacious.

### EXAMPLE 2

To further evaluate the minimum antigen level required for protection against an S. equi challenge with the present composition containing saponin, a dose titration study was conducted.

Test Animals

Sixty-three S. equi negative, clinically healthy horses were utilized in this study. The horses were screened by nasal isolation and ELISA against S. equi M-protein. All horses were from South Dakota, U.S.A. having the age of nine months or younger at the time of the first vaccination. The horses were housed in an isolation facility for the duration of the study under veterinary care and were fed a standard commercial diet with water and food available ad libidum. The vaccinates and controls were housed separately during the vaccination period. All horses were housed together two days before challenge until the end of the study. The housing complied with applicable animal welfare regulations. No animals were treated with antibiotics or anti-inflammatory drugs during the duration of the study.

Vaccine Composition and Vaccination Schedule

The preparation containing vaccine organisms used in this study was produced at the highest passage level allowed (i.e., MS+5) as described in Example 1. The lyophilized S. equi preparation was reconstituted with deionized water containing 2.5 mg/ml of saponin. Adjustments to obtain the target viable count were made as described in Example 1. The vaccine was stored at 2-7° C. The commercial vaccine was used according to the manufacturer's instructions.

The horses were randomly assigned to 6 groups. The randomization process was completed as described in Example 1. The experimental design is outlined in the following table:

| | No. | | |
|---|---|---|---|
| Group | of Horses | First Dose | Second Dose |
| 1 | 10 | 1 × 10⁵ CFU/dose | 2 × 10⁴ CFU/dose |
| 2 | 9 | 1 × 10⁶ CFU/dose | 2 × 10⁵ CFU/dose |
| 3 | 11 | 1 × 10⁷ CFU/dose | 2 × 10⁶ CFU/dose |
| 4 | 11 | 1 × 10⁸ CFU/dose | 2 × 10⁷ CFU/dose |
| 5 | 11 | Commercial (Bayer) | Commercial (Bayer) vaccine |
| | | vaccine | |
| 6 | 11 | No Vaccine | No Vaccine |

All vaccinates received two vaccinations three weeks apart. The vaccine composition was administered intranasally to horses in Groups 1-4. All such vaccinations were administered intranasally with a syringe connected to a flexible tubing of five inches in length. The first vaccination was administrated into the left nostril and the second vaccination was administrated into the right nostril. The commercial vaccine contained adjuvanted S. equi extract and was administered intramascularly using a needle and a syringe. The control horses were not vaccinated.

Challenge and Observation Procedure

Twenty-one days after the second vaccination, each of the 52 vaccinated and 11 control horses were challenged intranasally with a virulent S. equi organism (isolate CF32), which was prepared and stored as described in Example 1. One ml of the challenge culture was administered per nostril. The viable counts of the challenge culture were determined to be 5.2×10⁷ CFU/ml and 4.8×10⁷ CFU/ml prior to and post challenge, respectively.

The animals were observed daily from −1 days to 0 days post challenge (DPC) to establish a baseline and 1 to 21 DPC (excluding 18 and 20 DPC) for various clinical signs. Animals were observed additionally on 23, 26, 28, 33 and 35 DPC.

Whole Blood Samples and Hematology Evaluation

Five ml samples of whole blood were collected daily on −1 to 23 DPC for analysis by the Abbot CeH-dyne blood counter for white blood cell count (excluding 17, 18, 20 and 22 DPC). Baseline measurements for each horse were established as the average of the counts on −1 to 0 DPC.

Clinical Scoring System

A system used to score clinical data was as in Example 1, except that pyrexia was scored as 1 point for temperatures between 103.0 and 104.0° F., and as 2 points for temperatures between 104.0 and 105.0° F.

Statistical analysis was conducted as described in Example 1.

### RESULTS

Clinical Observations

The daily clinical signs (from −1 DPC to 35 DPC) and daily rectal temperatures (from −1 DPC to 35 DPC) for each horse were observed. After S. equi challenge, horses showed variable clinical signs, including fever, mucopurulent nasal discharge, and enlarged lymph nodes. Specifically, 73% of control horses (Group 6) developed ruptured abscesses and 81% of horses vaccinated intramascularly with the Bayer adjuvanted extract (Group 5) developed ruptured abscesses. However, only 36% of the horses in group 4 (1×10⁸ CFU/dose) developed ruptured abscesses, demonstrating a great reduction of disease incidence in this group in comparison to Groups 5 and 6. These findings support the surprising discovery described in the present application, i.e., the composition of the present invention is capable of inducing satisfactory protection against strangles in horses while the commercially available adjuvanted S. equi extracts were not as effective. The disease incidence in Groups 1, 2 and 3 (i.e., lower titer groups) was similar to that in the control group.

An average clinical score of 67.4 was observed for the control group, 66.2 points for Group 1, 52.8 points for Group 2, 53.6 points for Group 3, 23.6 points for Group 4, and 53.9 points for Group 5.

The total number of horses that died due to challenge or were euthanized for humane reasons (due to moribund state resulting from challenge) through the 35-day observation period was 1 of 10 (10%) for Group 1, 1 of 9 (11%) for Group 2, 1 of 11 (9%) for Group 5, and 2 of 11 (18%) for control (Group 6). Staff veterinarians with no knowledge of treatment groups made the decision on animals requiring euthanasia.

Daily white blood cell counts were observed. An average daily WBC counts for the control and Group 5 horses were consistently higher than those in Group 4 from 10 DPC to 15 DPC. Statistically, a significant difference (p<0.05) was seen when daily WBC counts of the vaccinates in Group 4 on 10-15 DPC were compared to the horses in Group 5 and the control. The average WBC counts in group 1, 2 and 3 were not statistically significant over the counts in the control group.

Conclusion

The composition of the invention can protects intranasally vaccinated horses against a virulent S. equi challenge when the second vaccination dose was at least 2×10⁷ CFU. However, it is possible that the composition of the present invention containing S. equi in the amount less than 2×10⁷ and a more potent immunostimulant(s) for stimulating mucosal immunity may provide satisfactory immunity and protection of intranasally vaccinated horses against strangles. In addition, results from this study demonstrated that the composition of the invention provides better protection than a commercially available adjuvanted S. equi extract composition for intramuscular vaccination.

### EXAMPLE 3

The objective of this study was to demonstrate the safety of the composition of the present invention when administered to horses by evaluating reversion to virulence of attenuated S. equi strain.

Test Animals

Fifty-six S. equi negative, clinically healthy weanling Quarter horses were utilized in this study. The horses were screened by nasal isolation and ELISA against S. equi M-protein. Thirty-two horses were from Myrtle, Minnesota and 24 horses were from Lake Mills, Iowa. All horses were 9-11 months old at the time of vaccination. The horses were housed in an isolation facility for the duration of the study under veterinary care and were fed a standard commercial diet with water and food available ad libidum. The vaccinates and contact controls were housed together during the study period.

Test Composition and Vaccination Schedule

The S. equi strain 709-27 used in this study was produced at the lowest production passage level (MS+1). The lyophilized strain composition was reconstituted with deionized water containing 2.5 mg/ml of saponin. Adjustments to obtain the target viable count were made by diluting the reconstituted vaccine with an adjustment diluent (75 percent Todd Hewitt broth, 25 percent SGGK stabilizer, and 2.5 mg saponin per ml). The vaccine was stored at 2° C. to 7° C. prior to use.

The horses were randomly assigned to each experimental group. The randomization process was performed as described in Example 1. The experimental design is outlined in the following table:

| | | Vaccination | |
|---|---|---|---|
| Passage | Number of Horses | Dose (approximate | Control Horses |
| Number | Vaccinated | CFU/dose) | (not vaccinated) |
| 1 | 20 in Group A | 9.99 × 10⁸ CFU/dose | |
| | 10 in Group B | 1.06 × 10¹⁰ CFU/dose | 3 |
| 2 | 5 | 1.4 × 10³ CFU/dose | 3 |
| 3 | 5 | Blind Inoculum | 3 |
| 4 | 5 | Blind Inoculum | 3 |

Horses in the first passage received a 3 ml dose intranasally, 1.5 ml/nostril. Horses in each subsequent passage received a 2 ml dose intranasally, 1.0 ml/nostril. All vaccinations were performed using a syringe equipped with a five inch catheter.

In the first passage, group A horses were inoculated with a composition containing 9.99×10⁸ CFU/dose and group B horses were inoculated with a composition containing 1.06×10¹⁰ CFU/dose. Nasal swab samples containing S. equi were collected from the horses in the first passage and split into two fractions. One fraction was used for testing on the day of collection. The other fraction was frozen at −70° C. All frozen nasal swab samples collected from the first passage were thawed, pooled and used for the inoculum of the second passage. Since no S. equi was identified in swab samples of horses in passage 2, two blind passages were conducted. To prepare the inoculum for the subsequent passages (passage 3 and passage 4), all nasal swab samples collected from all inoculated horses in the previous passage were thawed, pooled and concentrated by centrifugation (13,000 g for 40 minutes). The pellet was resuspended with an adjustment diluent and used at 2.0 ml per dose for the subsequent passage.

Clinical Observations

All horses were monitored for rectal temperature and observed for clinical signs associated with infection of S. equi, including but not limited to respiratory distress and local or systemic lymph node enlargement. The observations were conducted two days before each inoculation to establish a baseline and continued daily for up to 14 days following each inoculation.

As noted above, a blind study was conducted in passage 3 due to the absence of S. equi from the passage 2 horses. An additional blind passage (passage 4) was performed according to the USDA standards. The observation period of the blind passages was shortened to 7 days following inoculation.

Sample Collection and Testing

All animals were bled (maximum 15 ml whole blood) for serum at the day of vaccination, and days 7 and 14 post vaccination. The sera were tested for antibodies against S. equi heat extracted antigens by ELISA as described in Example 1.

Nasal swabs were collected from each horse 2 days prior to each inoculation and daily for at least 7-14 days post vaccination and were processed as described in Example 1.

Comparison of Master Seed, MS+1 and S. equi Isolate from Last Passage by SD-PAGE

Whole cell lysate prepared from the S. equi isolate in the first passage was analyzed for protein profile using SDS-PAGE and compared with Master seed, MS+1 and control samples (S. equi virulent strain CF-32, S. zooepidemicus and S. equisimilis). Briefly, samples were diluted in reducing buffer containing 0.3 M Tris-HCL, 5% SDS, 50% glycerol and 100 mM dithiothreitol (Pierce) and boiled for 10 minutes. Approximately 20 μg of protein was loaded in each well. Electrophoresis was carried out using slab gel with a 4% stacking gel and a 10% separating gel. After electrophoresis, the gel was fixed and stained with Coomassie blue.

Clinical Scoring System

The following scoring system was used:

| | | |
|---|---|---|
| (a) | Coughing | (1 Point/Day) |
| (b) | Nasal discharge | |

| | | |
|---|---|---|
| (1) | Serous | (1 Point/Day) |
| (2) | Mucopurulent | (2 Points/Day) |

| | | |
|---|---|---|
| (c) | Depression | (1 Point/Day) |
| (d) | Pyrexia | 1 Point/≧103° F./Day |
| | | 2 Points/≧104° F./Day |
| | | 3 Points/≧105° F./Day |

Temperature must be 1° F. above baseline before score can be assigned.

| | |
|---|---|
| (e) | Enlargement of Lymph nodes |

| | | |
|---|---|---|
| (1) | Head and neck areas | (3 Points/Day) |
| (2) | Systemic | (5 Points/Day) |

| | | |
|---|---|---|
| (f) | Death | (100 Points-One Time Score) |

Statistical Analysis

The level of significance for each statistical analysis was set at p<0.05. All analysis were performed as described in Example 1.

### RESULTS

Clinical Observations

Two horses from passage 1, #115 (group B) and #118 (group A) were removed from this study due to antibiotic treatment for a wound infection. Another two horses from passage 1, #119 (group A) and #80 (control) were euthanized due to displacement of the large colon and peritonitis which may have resulted from rectal perforation caused by a temperature probe.

The daily clinical signs of remaining horses from passage 1 to passage 4 were observed. After each inoculation, some horses in both vaccinate groups and control groups showed minor clinical signs, including serous or mucopurulent nasal discharge and ocular discharge. Horse #95 (Group A) in passage 1 showed a transient swollen lymph node (from 3 DPV to 9 DPV). The swollen lymph node had regressed by 10 DPV. Both the vaccinates and the controls in passage 2 had slight mucopurulent nasal discharge which may have resulted from a sudden change in temperature due to a snow storm that occurred during this phase of the study.

The daily temperature of each horse in passage 1 to passage 4 were recorded. Following first inoculation, some horses in both vaccinate groups and control groups showed transient fever (from 103° F. to 104.6° F.) with no other clinical signs. Horse #121 (group A) had a temperature of 105.4° F. on 5 DPV with no other clinical signs. From passage 2 to passage 4, most horses in both vaccinate groups and control groups did not have fever after inoculation. Horse #166 (passage 3) had a temperature of 103.2° F. at 1 DPV and horse #178 (passage 4) had a temperature of 103.2° F. at 4 DPV. Horse #180 (control) in passage 4 had a fever (104.1° F.) at 2 DPV due to a seroma development on the chest. Some horses were excitable, wild and difficult to handle during the observation period. The excitement and wild behavior of the horses could have caused the high temperatures seen in this study.

The daily and total clinical scores of each group in passages 1 to 4 were observed. The average score of group A in passage 1 was 4.5 points, group B was 3.2 points and control group was 3.5 points. Statistically, no significant difference was seen when comparing the total clinical scores of either vaccinate group to the control group (p>0.05). In passage 2, the average score of vaccinate group was 5.4 points and the control group was 5.3 points. In passage 3, the average score of vaccinate group was 1.8 points and the control group was 3.3 points. In passage 4, the average score of the vaccinate group was 1.6 points and control group was 2.0 points. Statistically, no significant difference was seen when comparing the total clinical scores of either vaccinate group and the control group in each passage (p>0.05).

Additionally, no significant difference was seen when comparing the total clinical score of vaccinate group between passages (p>0.05).

Serological Responses

Serum IgG titer of the vaccinated and control horses from passage 1, as determined by the ELISA test. At 0 DPV1, all vaccinated horses had ELISA titers≦1:160. Most of the horses remained seronegative (ELISA titers≦1:160) throughout the observation period, except that three horses (#65, #72 and #78) in group A seroconverted (≧folds increase) by 14 DPV. Titers of <1:160 were considered as 1:80 for the purpose of analysis. Statistically, neither of the vaccinated groups had a significant difference in ELISA titer throughout the study period when compared to the controls (p>0.05).

The ELISA titer of the vaccinated and control horses from passage 2 to passage were also obtained. No seroconversion was identified in vaccinated or control horses in passage 2, passage 3 and passage 4 throughout the observation period. Statistically, neither of the vaccinated groups had a significant difference in ELISA titer throughout the study period (passage 2, passage 3 and passage 4) when compared to the controls (p>0.05).

Additionally, no significant difference was seen when comparing the ELISA titer between each passage (from passage 1 to passage 4, p>0.05).

S. equi Shedding After Vaccination

One day following vaccination, S. equi was identified from three horses (#65, #82 and #110) in group A and one horse (#77) in group B. The remaining horses were free of detectable shedding throughout the observation period. No statistically significant difference was seen when comparing daily shedding incidence between either vaccinate group to the control group (p>0.05).

No S. equi shedding was identified from any horse from passage 2 to passage 4 throughout the observation period.

Statistically, no significant difference was seen when comparing the daily shedding incidence of vaccinates between each passage (from passage 1 to passage 4, p>0.05).

Therefore, a low level of shedding was identified only from the vaccinates in passage 1. Moreover, the duration of shedding was short (only 1 day after vaccination) even for the horses vaccinated with 20 fold the expected maximum field dose (group B in passage 1). The quantity and duration of shedding did not increase between the first and the last passage. No shedding was identified from any of the controls.

Comparison of Master Seed, MS+1 and S. equi Isolate from Last Passage by SDS-PAGE

Protein profiles of vaccine strain Master Seed, MS+1 and S. equi isolate from passage 1 as well as control samples, S. equi virulent strain CF-32, S. equisimilis and S. zooepidemicus were compared by SDS-PAGE. The protein profiles from Master Seed and MS+1 were similar to that of the S. equi last passage isolate, indicating there was no change in protein profiles among the Master Seed, MS+1 and the last passage isolate. Result from the SDS-PAGE demonstrated that there was no detectable protein profile difference between Master Seed, MS+1 the S. equi isolate from last passage.

Conclusion

The data obtained from the above described reversion to virulence study demonstrates that the S. equi strain used in the composition of the invention did not revert to virulence when it was inoculated intranasally to susceptible horses at the lowest production passage level (MS+1) and then repeatedly backpassaged in susceptible horses. Statistically, there was no significant difference (p>0.05) of clinical score between the vaccinate and the control groups in each passage or between passages. These findings establish the safety of administering the composition of the present invention to horses.

What is claimed:

1. A composition for providing protective immunity against Streptococcus equi infection following S. equi challenge comprising a live non-encapsulated attenuated Streptococcus equi in combination with an immunostimulant that stimulates mucosal immunity when administered to nasal or oral mucosa.

2. The composition of claim 1, wherein said Streptococcus equi is strain 709-27 (ATCC 53186).

3. The composition of claim 1, wherein the immunostimulant is saponin or a bacterial toxoid.

4. The composition of claim 3, wherein the bacterial toxoid is cholera toxin subunit.

5. The composition of claim 3, wherein the immunostimulant is saponin and the saponin is present in an amount of from about 1 to about 10 mg/ml of the composition.

6. The composition of claim 5, wherein said saponin is in an amount of from about 2.5 to about 7 mg/ml of said composition.

7. The composition of claim 3 wherein the saponin is Quil A.

8. A method of stimulating an immune response to Streptococcus equi comprising contacting cells of the nasopharyngeal mucosa of an equine with a composition comprising a live, non-encapsulated, attenuated Streptococcus equi in combination with an immunostimulant that stimulates mucosal immunity, wherein said immunostimulant is saponin, and wherein the composition provides protective immunity against Streptococcus equi infection following Streptococcus equi challenge.

9. The method of claim 8, wherein said Streptococcus equi is strain 709-27 (ATCC 53186).

10. The method of claim 8, wherein the composition is administered in two doses prior to Streptococcus equi challenge, wherein the second dose is administered about 10 days to six weeks after the first dose.

11. The method of claim 10, wherein the second dose is administered about three weeks after the first dose.

12. The method of claim 10, wherein the first dose contains an amount of Streptococcus equi of from about 1.5×10⁷ to about 1.0×10⁸ Colony Forming Units (CFU).

13. The method of claim 10, wherein the second dose contains an amount of Streptococcus equi of about 2.0×10⁷ Colony Forming Units (CFU).

14. The method of claim 8, wherein the saponin is present in amount of about 2.5 mg/ml.

15. A method for preventing at least one of the symptoms associated with Streptococcus equi infection in equine comprising administering to the nasal or oral mucosa of said equine an effective amount of a composition comprising a live, non-encapsulated, attenuated Streptococcus equi in combination with an immunostimulant that stimulates mucosal immunity, wherein said immunostimulant is saponin, and wherein the composition is suitable for providing protective immunity against Streptococcus equi infection following Streptococcus equi challenge.

16. The method of claim 15, wherein said Streptococcus equi is strain 709-27 (ATCC 53186).

17. The method of claim 15, wherein the composition is administered in two doses prior to Streptococcus equi challenge, wherein the second dose is administered about 10 days to six weeks after the first dose.

18. The method of claim 17, wherein the second dose is administered about three weeks after the first dose.

19. The method of claim 17, wherein the first dose contains an amount of Streptococcus equi of from about 1.0×10⁶ to about 1.0×10⁸ Colony Forming Units (CFU).

20. The method of claim 17, wherein the second dose contains an amount of Streptococcus equi of from about 2.0×10⁵ to about 2.0×10⁷ Colony Forming Units (CFU).
